(19) United States
(12) Patent Application Publication
(10) Pub. No.: US 2007/0008493 A1
(43) Pub. Date: Jan. 11, 2007

(54) Method and vision testing apparatus for determining the necessity of a vision aid during darkness and/or twilight as well as a set of vision aids

(76) Inventor: Timo Kratzer, Aalen (DE)

Correspondence Address: WALTER OTTESEN, PO BOX 4026, GAITHERSBURG, MD, 20885-4026, US

(21) Appl. No.: 11/484,667

(22) Filed: Jul. 12, 2006

Related U.S. Application Data
(63) Continuation of application No. PCT/EP04/14278, filed on Dec. 15, 2004.

(30) Foreign Application Priority Data
Jan. 24, 2004 (DE) 10 2004 003 688.8

(51) Int. Cl. A61B 3/00; A61B 3/10

(52) U.S. Cl. 351/246; 351/211; 351/221

(57) ABSTRACT

A method and a vision test apparatus determine the necessity of a vision aid during darkness and/or twilight. Also, a set of vision aids are referred to. The daylight defective vision is measured at at least one value of high luminance=10 cd/m² and the night defective vision is measured at at least one value of low luminance <10 cd/m². Thereafter, the difference between the daylight defective vision and the night defective vision is determined and, when a defined value is exceeded, the recommendation for using a vision aid during darkness and/or twilight is outputted.

### CROSS REFERENCE TO RELATED APPLICATIONS

This application is a continuation application of international patent application PCT/EP 2004/014278, filed Dec. 15, 2004, and claiming priority from German application 10 2004 003 688.8, filed Jan. 24, 2004, and the entire content of both applications is incorporated herein by reference.

### FIELD OF THE INVENTION

The invention relates to a method and a vision testing apparatus for determining the necessity of a visual aid for darkness and/or twilight as well as a set of visual aids including spectacles and/or contact lenses.

### BACKGROUND OF THE INVENTION

Vision defects of the eyes are generally corrected with spectacles or contact lenses in order to increase the vision capacity, that is, the quality of vision. For this purpose, the refractive indices, such as sphere, cylinder and position of the axis of the spectacle lens or of the contact lens, which are optimal for improving vision, are determined in a subjective or objective measurement method. Refractive measuring methods are known such as wavefront detection as described in U.S. Pat. 6,511,180, which not only permit determination of the above-mentioned values of sphere, cylinder and axis, but also the determination of higher order aberrations.

The effects of the aberrations of higher order are dependent upon the opening of the pupil of the eye. The magnitude of the pupil opening is, inter alia, influenced by the brightness of the ambient, medications, age and illnesses of the person examined. The pupil opening in daylight fluctuates between 2.5 and 3.5 mm for a healthy adult and reduces with increasing age. The pupil opening is greater for decreasing luminous densities and the effects of the aberrations of higher order increase.

U.S. Pat. No. 5,638,082 discloses a vision testing system wherein optotypes are generated by means of a light-emitting display screen. This arrangement can so influence the light coming from the display screen that optotypes or parts thereof can be seen in a targeted manner only by one eye or both eyes. By varying the luminous intensity and contrast of the display screen, the contrast sensitivity and the adaptation behavior can be determined. A method for the targeted determination of the necessity of night spectacles is, however, not disclosed.

U.S. Pat. 5,870,168 discloses a vision testing apparatus for the subjective testing of twilight vision. This vision testing apparatus includes several optotypes, which can be switched into the beam path as desired, and a diaphragm light source in a closed housing. In this apparatus, the daylight visual acuity or decremented vision value under twilight conditions can be additionally determined. No connection is made here between the determined daylight visual acuity and the twilight visual acuity. The particular test person and the optometrist are left to their own devices for evaluating the determined values. A determination of aberrations of higher order, the effects of which increase especially with increasing darkness, is not undertaken.

### SUMMARY OF THE INVENTION

It is an object of the invention to provide a method and a testing apparatus with which the necessity as to a visual aid for darkness and/or twilight is determined for persons having emmetropic vision and persons having defective vision. Additionally, it is an object of the invention to provide a set of vision aids which enables a person having defective vision to see well during daylight as well as at night.

The method of the invention is for determining the need for a visual aid in darkness and/or twilight. The method includes the steps of: measuring the defective daylight vision at at least one value of high luminous density >10 cd/m²; measuring the defective night vision at at least one value of low luminous density <10 cd/m²; and, then, determining the difference between defective daylight vision and defective night vision and outputting a recommendation for the use of a visual aid for darkness and/or twilight when a defined value of the difference is exceeded.

With the method of the invention, a determination is made as to the necessity of a visual aid in darkness and/or in twilight. According to a feature of the invention, the defective daylight vision is measured with at least one value of high luminous intensity <10 cd/m² and the defective night vision is measured with at least one value of low luminous intensity <10 cd/m². Thereafter, the difference between the defective daylight vision and the defective night vision is determined and, when a defined value is exceeded, the recommendation to use a visual aid during darkness and/or twilight is outputted.

The above method can also be used to determine the defective daylight vision and the test person is thereafter given the determined values. In this way, spectacles can be recommended to the test person for daylight. Depending upon the determined defects of vision, day spectacles and night spectacles or only day spectacles or only night spectacles can be fitted for the test person.

The defective daylight vision is preferably measured at a value of high luminous intensity≧10² cd/m², especially, in the range of 10³ cd/m² to 10⁵ cd/m². The given range corresponds to the brightness to which a person is normally subjected during the day in central Europe. In this way, sunny days as well as cloudy days are included. Luminous intensities can be selected which are adapted to the respective external conditions. The defective daylight vision is determined when there is at least one value in this range of luminous intensities. For a more precise determination of the defective daylight vision, the particular vision defect can also be determined at several luminous intensities and then the defective daylight vision is determined via suitable averaging. For example, the sphere (refractive index values) can be averaged purely arithmetically and the cylinder with axis position can be averaged vectorially.

The defective night vision is preferably measured at a value of low luminous intensity<10⁻² cd/m², especially in the range of 10⁻² cd/m² to 10⁻⁵ cd/m². Here too, the range can be so selected that it is adapted to the requirements of the wearer. For this, the average range of the luminous intensities can be determined to which, for example, a driver of automobiles is subjected at night, if required, also at twilight. In this way, an intense twilight, illuminated streets at darkness as well as also pitch black nighttime are detected. The defective night vision is determined for at least one value in this range of luminous intensities. For a more precise determination of the defective night vision, the particular vision defect can be determined at several light intensities and then the defective night vision can be determined by suitable averaging.

The output of a recommendation for use of a night vision aid is especially of interest to those persons who are underway because of their occupation in street traffic during darkness or possibly also during twilight. It is known that automobile drivers, for whom a vision defect was determined, should wear a visual aid, that is, spectacles or contact lenses when driving an automobile. This is noted in the driver's license for persons obtaining a driver's license for the first time and is therefore a requirement. No attention is given at the present time to the fact that some persons exhibit emmetropy during daylight, that is, have normal vision and are myopic during darkness, often <−0.5 dpt and less often <−1.0 dpt. This is often increased for persons who already exhibit myopia. This vision defect can be a reason for the high rate of accidents during twilight and at night.

Internal experiments have shown that for one group of the test persons the night vision acuity remains the same in comparison to the day visual acuity. In a further group of the test persons, the visual acuity at night deteriorated by at least one increment of visual acuity. Often, spherical changes of the defective vision were present of 0.5 to 1.0 dpt. Also, changes of the cylinder values and axial values could be observed. In a few test persons, the visual acuity improved at night compared to the visual acuity during the day.

The vision defects during the day and at night can be determined in accordance with a preferred embodiment of the invention with, in each case, an objective measurement method, for example, a wavefront measurement. It is conceivable as an alternative or as an addition to provide a subjective measurement, for example, utilizing optotypes. To provide an improved contrast ratio, inverse optotypes can be utilized, that is, optotypes on a dark background, especially optotypes built up by LEDs. Good refraction values are obtained only at high contrast.

The defective day vision and/or defective night vision can preferably be determined using wavefront analysis. In addition, it is conceivable that the defective daylight vision is determined in accordance with the autorefractive method. For example, the defective day vision can be measured in accordance with wavefront analysis as well as in accordance with the autorefractive method. If different day vision defects are determined for a test person, the test person can select the result which is subjectively best for him or her.

According to the invention, spherical values can be determined for the day vision defects and the night vision defects. Also, a determination of the cylinder and the axis can be undertaken. Furthermore, aberrations of higher order, especially spherical aberration, coma and/or three-leaf aberrations can be determined. Three-leaf aberrations are aberrations of higher order and are described in the text by Harry Paul entitled “Lexikon der Optik”, published by Spektrum Akademischen Verlag (1999).

Usually, the defective day vision and thereafter the defective night vision is measured. It is understood that this procedure can proceed in the opposite sequence. For each measurement, it should be noted that the pupils have adapted to the particular luminous intensity. To determine the effect of the vision defects, especially vision defects of higher order, the pupil size can be measured, especially, in dependence upon the luminous intensity used.

According to an embodiment of the method of the invention, the recommendation to use a visual aid during darkness can include a suggestion and/or an offer for suitable spectacles and/or contact lenses.

In a further embodiment of the method of the invention, the recommendation of a visual aid to correct defective night vision is outputted if, during the determination of the difference between defective day vision and defective night vision, at least one of the following difference values is exceeded:

(a) for the spherical values: ≧¼ dpt;

(b) for the cylinder values: ≧¼ dpt;

(c) for the axis change: ≧7°; and, (d) for the mean sphere: ≧¼ dpt.

The axis change is dependent upon the cylinder change. The mean sphere is defined as “sphere+½ cylinder”. In lieu of “≧¼ dpt” for (a), (b) and/or (d), also “≧½ dpt” can be provided.

A recommendation can not only be outputted when the defective vision is worse at night but also when it has gotten better. Wearing a visual aid adjusted too much can also lead to irritation when looking.

For those test persons which are below the defined value, no such recommendation is outputted, that is, this group of persons can continue to do without spectacles or with the day spectacles.

It can be provided that after the measurement of the defective night vision and defective day vision, a subjective check of the night values can take place by means of optotypes for the test person at low luminous intensity. Corrective lenses in the form of spectacle lenses or contact lenses can be held in front of the person or seated on the person with the night values as well as with the day values.

In this way, the test person can himself or herself directly experience the difference between day spectacles and night spectacles. This can significantly increase the willingness to wear other spectacles at night and thereby reduce the danger in street traffic.

In a further embodiment of the method of the invention, the defective daylight vision can be determined by averaging with different values of high luminous intensity. Also, the defective night vision can be determined by averaging with different values of low luminous intensities.

Furthermore, it can be provided that after the measurement of the defective daylight vision and defective night vision, optotypes for viewing by the test person can be utilized for visualizing a specifically present difference between defective daylight vision and defective night vision. The sharpness of the optotype varies in dependence upon the determined defective daylight vision and/or defective night vision.

For this purpose, and after the measurement of the defective night vision and ideally at a time point at which the test person still has wide open pupils, the vision testing apparatus of the invention can show to the test person an image for visualizing his or her defective vision at darkness, for example, the image can be an eye chart, an image of a landscape, an image of an expressway at night. This image first shows the sharpness that the test person can achieve at darkness with his or her normal daytime spectacles. For this reason, this image is to be shown more or less blurred to many test persons.

Test persons who have normal vision during daylight are alternatively shown an image which shows the acuity which the test person can achieve without spectacles in darkness. Here too, a blurred image is to be shown to many test persons.

Thereafter, the test person is shown the same image with improved sharpness which can be achieved with night vision spectacles. In this way, even skeptical test persons, which have deemed it unnecessary to have a second set of spectacles, can be moved to acquire such spectacles for the purpose of safety at least for use when driving an automobile.

In a further embodiment of the method of the invention, it can be provided that different metrics or procedures are used for measuring the defective night vision. For measuring the defective night vision, the defective vision can be determined with different metrics, such as Strehl ratio of the point spread function (PSF), entropy of the PSF, contained energy of the PSF, modulation transfer function (MTF). Finally, it can be provided that the particular vision defect is measured at various stages of twilight. This can be so integrated into the recommendation for use of the visual aid that the test person is recommended to wear a first spectacles during daylight, a second spectacles during twilight and a third spectacles at night. For specific occupations, a still finer gradation can be purposeful.

The vision test apparatus of the invention is for determining the necessity of a visual aid at darkness and/or at twilight. According to a feature of the apparatus of the invention, a wavefront detector is provided for measuring the defective daylight vision and the defective night vision and an evaluation unit is provided for determining the necessity of night spectacles. The evaluation unit detects the values of the defective daylight vision and the defective night vision and, when a defined value is exceeded, the recommendation to utilize a visual aid at darkness is outputted. The output can, for example, take place via display or via a printer. The recommendation can comprise a simple “yes” or “no” or the precise values can be outputted for the night spectacles and/or day spectacles.

The vision test apparatus can include a wavefront detector for the objective determination of vision defects and/or optotypes for the subjective determination of vision defects. With the wavefront detector, not only vision defects of the second order such as sphere, cylinder and axis (SCA) can be detected, but also vision defects of higher order. Elements such as a dimmer can be provided for controlling luminous intensity.

A unit can be provided for measuring pupil size in the vision test apparatus of the invention.

The set of vision aids according to the invention, can include spectacle lenses and/or contact lenses. According to a feature of the invention, a first visual aid is provided for compensating defective vision during daylight and at least a second visual aid for compensating a defective vision at darkness and/or twilight.

For example, a set of visual aids can include a first visual aid for compensating a vision defect during darkness, a second visual aid can be provided for compensating defective vision at twilight and a third visual aid can be provided for compensating defective vision during daylight.

The first visual aid can comprise spectacle lenses and contact lenses as required. The second visual aid can also comprise spectacle lenses and contact lenses as required. The spectacle lenses can be made of glass as well as of plastic. It is conceivable to combine the first and second visual aids with each other for optimal vision during darkness. Accordingly, for daylight, spectacles having −2 dpt can be provided. At night, a test person has, however, −3 dpt. This test person can then wear a clip on his or her spectacles. This clip includes for one or both eyes a lens for compensating the remaining vision defect. It is also conceivable to utilize contact lenses during the day. During darkness, the contact lenses remain on the eye. Additionally, spectacles can be worn to compensate for the remaining defective vision. The particular vision aid can have refractive and/or diffractive structures.

### BRIEF DESCRIPTION OF THE DRAWING

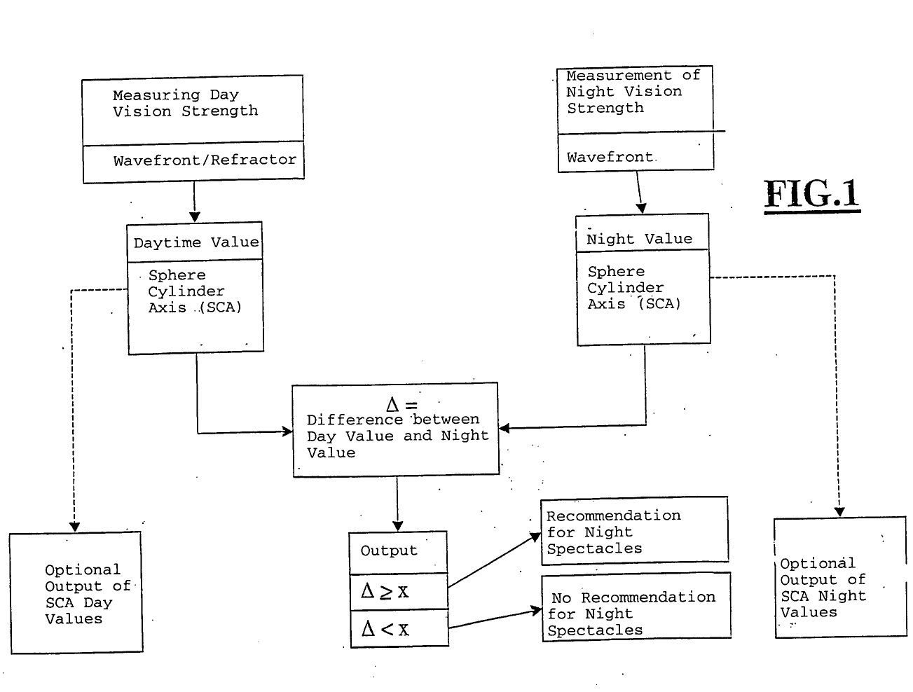

The invention will now be described with reference to the single figure of the drawing (FIG. 1) wherein the method and the apparatus of the invention are explained.

### DESCRIPTION OF THE PREFERRED EMBODIMENTS OF THE INVENTION

FIG. 1 schematically shows a preferred method sequence for determining the need for night spectacles. A vision test apparatus is provided which operates utilizing a wavefront mode and, if needed, also utilizing an autorefractor mode. A test person is screened off from ambient light and looks into the test apparatus with one eye or with both eyes. The eye or both eyes to be examined are illuminated. In this way, the pupil reduces to a value of approximately 2 to 4 mm, for example, approximately 3.5 mm. Now, the measurement of the defective daylight vision can take place. The measurement can take place exclusively in the wavefront mode. It can, however, selectively take place in the autorefractor mode. If required, both modes can be used. The values of the defective daylight vision for, for example, sphere, cylinder and axis are determined, and can be presented to the test person, the ophthalmologist, the optometrist or another cognizant person for viewing.

In a further step, the defective night vision is measured. However, first the defective night vision and thereafter the defective daylight vision can be determined. For determining the defective night vision, the eyes of the test person are not illuminated or illuminated only slightly. The pupils widen, for example, to 6 mm. A wavefront measurement is made. The determined values for the defective night vision can likewise be outputted to the test person, the ophthalmologist or other cognizant person at the location of testing. It is also conceivable not to output the determined values for the defective night vision and/or the defective daylight vision but to use these values internally in the system in order to determine the difference from the values for the defective night vision and the values for the defective daylight vision and, when a defined value is exceeded, for example, at sphere ≧0.5 dpt, the recommendation of a visual aid for darkness or twilight can be outputted. The determined values for defective night vision and/or the defective daylight vision can be transmitted directly to the manufacturer of the spectacle glass. For this purpose, the values can already be converted into the usual dimensions, for example, sphere =−3 dpt or can be transmitted as a wavefront, preferably in arrow elevations or as Zernike coefficients. In the last case, the conversion can take place at the manufacturer.

It is understood that the foregoing description is that of the preferred embodiments of the invention and that various changes and modifications may be made thereto without departing from the spirit and scope of the invention as defined in the appended claims.

What is claimed is:

1. A method for determining the need for a visual aid in darkness and/or twilight, the method comprising the steps of:
measuring the defective daylight vision at at least one value of high luminous density ≧10 cd/m²;
measuring the defective night vision at at least one value of low luminous density <10 cd/m²; and,
then, determining the difference between defective daylight vision and defective night vision and outputting a recommendation for the use of a visual aid for darkness and/or twilight when a defined value of said difference is exceeded.

2. The method of claim 1, wherein the defective daylight vision is measured at a value of high luminous density ≧10² cd/m².

3. The method of claim 1, wherein the defective daylight vision is measured in the range of 10³ cd/m² to 10⁵ cd/m².

4. The method of claim 1, wherein the defective night vision is measured at a value of low luminous density <10⁻² cd/m².

5. The method of claim 1, wherein the defective night vision is measured in the range of 10⁻² cd/m² to 10⁻⁵ cd/m².

6. The method of claim 1, wherein the defective daylight vision and/or the defective night vision is determined by means of a wavefront analysis.

7. The method of claim 1, wherein the defective daylight vision is determined in accordance with the autorefractor method.

8. The method of claim 1, wherein the spherical values are determined for the defective daylight vision and/or the defective night vision.

9. The method of claim 1, wherein the cylinder and the axis are determined for the defective daylight vision and/or the defective night vision.

10. The method of claim 1, wherein, for the defective daylight vision and/or the defective night vision, aberrations of higher order are determined including spherical aberration, coma and/or three-leaf aberrations.

11. The method of claim 1, wherein, for the determination of the effect of vision defects including vision defects of higher order, the pupil size is measured in dependence upon the luminous density utilized.

12. The method of claim 1, wherein the recommendation for the use of a visual aid for darkness and/or twilight includes a suggestion and/or an offer for suitable spectacle lenses and/or suitable contact lenses.

13. The method of claim 1, wherein the recommendation of a visual aid to correct a vision defect is outputted for darkness and/or twilight when, in the determination of the difference between defective daylight vision and defective night vision, at 5 least one of the following difference values is exceeded:
(a) for the spherical values ≧¼ dpt,
(b) for the cylinder values ≧¼ dpt,
(c) for the axis change ≧7°,
(d) for the mean sphere ≧¼ dpt.

14. The method of claim 1, wherein after the measurement of the defective daylight vision and the defective night vision, a subjective test of the night values takes place by means of optotypes for the test person and corrective lenses are held in 5 front of the test person in the form of spectacle lenses with night values as well as with day values or in the form of seated contact lenses with night values as well as with day values.

15. The method of claim 1, wherein the defective daylight vision is determined with different values of high luminous density via suitable averaging.

16. The method of claim 1, wherein, after the measurement of the defective daylight vision and defective night vision, for the visualization of a specific present difference between defective daylight vision and defective night vision, optotypes are utilized for viewing by the test person and the sharpness of these optotypes is varied in dependence upon the determined defective daylight vision and/or defective night vision.

17. The method of claim 1, wherein the defective night vision is determined at different values of low light density via averaging.

18. The method of claim 1, wherein different metrics are utilized for measuring the defective night vision.

19. The method of claim 1, wherein the defective vision is measured in each of different stages of twilight.

20. A vision testing apparatus for determining the need of a visual aid in darkness and/or twilight, the apparatus comprising:
a wavefront detector for measuring the defective daylight vision and the defective night vision;
an evaluation unit for determining the need for night spectacles;
the evaluation unit including means for detecting the values of the defective daylight vision and the defective night vision; and,
means for outputting a recommendation for the use of a visual aid in darkness and/or in twilight when a defined value is exceeded.

21. The vision testing apparatus of claim 20, wherein the vision testing apparatus includes a wavefront detector for objective determination of defective vision.

22. The vision testing apparatus of claim 20, further comprising an autorefractometer for objective determination of defective vision.

23. The vision testing apparatus of claim 20, further comprising optotypes for subjective determination of defective vision.

24. The vision testing apparatus of claim 20, further comprising elements for controlling the luminous density.

25. The vision testing apparatus of claim 20, further comprising a unit for measuring pupil size.
